(19) United States
(12) Patent Application Publication
(10) Pub. No.: US 2004/0073097 A1
(43) Pub. Date: Apr. 15, 2004

(54) Method and device for substantially restricting the freedom of movement of a living being and examining the living being and use thereof for optical imaging

(76) Inventors: Arne Hengerer, Erlangen (DE); Marcus Pfister, Erlangen (DE); Heike Sieber, Bamberg (DE)

Correspondence Address: HARNESS, DICKEY & PIERCE, P.L.C., P.O.BOX 8910, RESTON, VA, 20195, US

(21) Appl. No.: 10/621,405

(22) Filed: Jul. 18, 2003

(30) Foreign Application Priority Data
Jul. 18, 2002 (DE) 10232679.7

(51) Int. Cl. A61B 5/00

(52) U.S. Cl. 600/300

(57) ABSTRACT

A method and a device are for stabilizing living beings, in order to examine a living being to be examined by way of an examination instrument during a predeterminable time period in an examination area. To this end, the living being is enclosed by way of at least two gates in the examination area during at least the predeterminable time period Δt needed for the examination. As such, anesthesia can be obviated.

The present application hereby claims priority under 35 U.S.C. §119 on German patent application number DE 10232679.7 filed Jul. 18, 2002, the entire contents of which are hereby incorporated herein by reference.

### FIELD OF THE INVENTION

The invention generally relates to a method and to a device for stabilizing and examining living beings.

### BACKGROUND OF THE INVENTION

It is well known to stabilize or substantially restrict the freedom of movement of living beings, especially animals, before they are subjected to an examination. This is done, for example, to prevent the examination results from being vitiated owing to movement of the living being, for example in the case of ultrasound images, x-ray images or CT images. Furthermore, when studying metabolic processes at the molecular level, for example in living domestic animals, optical imaging instruments are used together with contrast agents which fluoresce in the near infrared range. Here again the living being to be examined needs to be stabilized. These methods are employed in many fields of biology, medicine and pharmacy, for example to explain signal transduction paths, in gene therapy, in tumor research and for the development of new medications.

Until now, living beings have anesthetized and fixed for the duration of the examination in the case of corresponding examination methods. Unlike larger animals or humans, there is a problem with domestic animals. In particular, only a small number of anesthesia protocols are available. Thus, accurate dosing of the anesthetics turns out to be difficult.

The anesthetic dose depends on the age, sex and metabolic rate of the animal. Thus, each anesthesia constitutes an increased health risk for the living being to be examined, especially for a domestic animal to be examined.

Anesthetization furthermore requires close monitoring of the vital parameters, in order to avoid complications during the examination. For example, mice in the anesthetized state are no longer capable of controlling their body temperature themselves, so that they are susceptible to hypothermia. The body temperature of these animals is externally measured during the anesthesia and, for example, regulated by way of a heated bed or a heat lamp.

Intensive care of the living beings to be examined during the examination is, on the one hand, time-consuming since it needs to be carried out manually. Further, it is costly since additional instruments and medications are necessary. There is also an increased health risk for the sometimes very expensive subject animals.

Conventional methods by way of anesthesia cannot provide a high throughput of subject animals, since automation is difficult. Furthermore, the anesthetized animal does not necessarily represent the actual physiological state, which reduces the applicability of the results of a possibly artificial system to other organisms, as well as the meaningfulness of the results.

DE 21 14 958 discloses a livestock treatment compartment which consists of a frame with an entry grille and an exit grille for the livestock; the livestock can be held inside the compartment by way of an attachment. After the livestock has been introduced into the compartment, the exit is closed. Then, a locking bar is fitted to the entry in order to prevent the livestock from reversing out of the cubicle.

DE 31 46 446 discloses a method and a device for taking blood from a living subject animal. Here, the subject animal is mechanically held between two plates by way of gate-like elements. Tube-like and/or tunnel-like retaining devices are furthermore mentioned, in which a subject animal can be enclosed, for example in order to take blood from the tail protruding from the device.

### SUMMARY OF THE INVENTION

It is therefore an object of an embodiment the invention to avoid the disadvantages of the aforementioned methods and devices according to the generic type, and to provide an inexpensive and not very time-consuming stabilizing method and device, which allow a higher throughput of the living beings to be examined. The examination results when using the method or the device should furthermore not be vitiated and, if possible, should actually be more accurate than in the conventional systems.

The method according to an embodiment of the invention for substantially restricting the freedom of movement of the living being, in order to examine an living being by way of an examination instrument during a predeterminable time period in an examination area. The method uses an examination area in which the living being is enclosed during at least the predeterminable time period. To this end, in particular, two gates which close the examination area are preferably used. The examination area and the at least two gates are parts of an examination system which furthermore has an entry and an exit as well as, optionally, an introduction area and a discharge area.

The method according to one embodiment of the invention preferably includes the following steps:

1. The second gate, which faces the exit, is closed.

2. The living being is introduced through the entry.

3. As soon as the living being is in the examination area, which is already half-closed by the second gate, the first gate of the examination area, which faces the entry, is also closed.

4. The examination is started as soon as the living being is in the examination area.

5. The second gate is re-opened after having closed the first gate, and after the predeterminable time period has elapsed, so that the living being can leave the examination area, optionally via the discharge area and through the exit of the examination system.

The method according to an embodiment of the invention makes it possible to examine the living being in a very short time without anesthesia and without additional medications, by holding it in the examination area, in particular between the two gates, for a predetermined examination time period. By obviating anesthesia for the living being, in particular by obviating anesthesia for domestic animals, possible complications for the living being are avoided. This leads to an increase in the throughput of the living beings to be examined in the examination system, and hence to a cost reduction.

The contact between humans and subject animals is furthermore minimized in the type of examination of domestic animals according to an embodiment of the invention, so that the infection risk of the subject animal is reduced. This serves, on the one hand, to protect humans against infectious animals and, on the other hand, to protect animals susceptible to disease (for example nude mice) against human germs.

### BRIEF DESCRIPTION OF THE DRAWINGS

A preferred embodiment of the invention, and the device according to the invention, will be explained in more detail with reference to FIG. 1, wherein:

FIG. 1 schematically shows the arrangement of the device for immobilizing living beings.

### DETAILED DESCRIPTION OF THE PREFERRED EMBODIMENTS

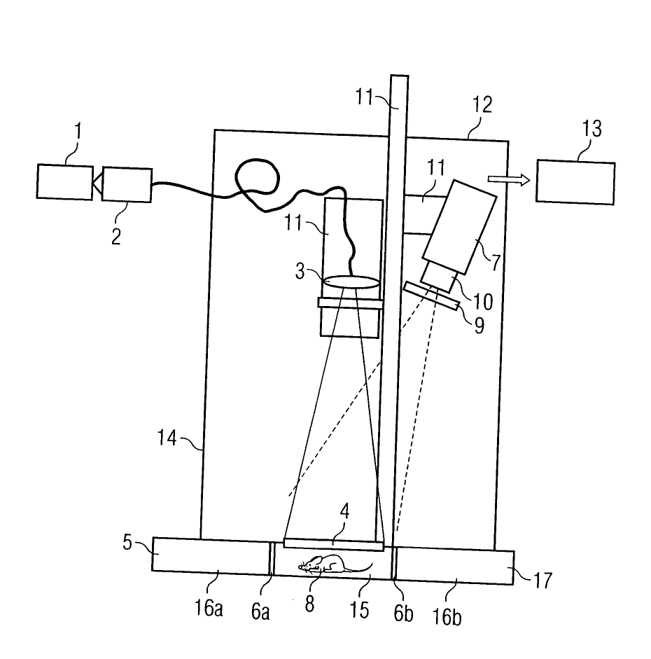

FIG. 1 schematically shows the arrangement of the device for stabilizing living beings, to substantially restrict the freedom of movement of the living being. The device includes an operatively coupled examination area 15 an examination instrument 7, in order to examine a living being 8. The living being may be examined during a predeterminable time period Δt in an examination system. The examination system and the examination instrument 7 are preferably arranged next to each other. According to the device according to an embodiment of the invention, the living being 8 is stabilized and enclosed in the examination area 15 during at least the examination time, which is preferably the predeterminable time period Δt.

The device according to an embodiment of the invention has at least two gates 6a, 6b, adapted to enclose the living being 8 in the examination area 15, and adapted to allow introduction and discharge of the living being 8. The examination system includes an entry, preferably an introduction area 16a, the examination area 15, preferably a discharge area 16b, and an exit 17. These elements preferably form an elongated tunnel, which even more preferably has a diameter that substantially restricts the freedom of movement of the living being 8 in all directions other than along the tunnel, in order to allow minimally distorted imaging of the living being 8.

In order to examine the living being by way of an optical imaging system, the examination system, or in any event the examination area 15 and the at least two gates 6a, 6b, are preferably made to be opaque. The optical examination instrument 7 is preferably used in connection with a window 4 coupled to the examination area 15. The window 4 is in any event transparent to the radiation needed for the examination (light rays, x-rays, electromagnetic waves etc.). For use of the immobilization device according to an embodiment of the invention in an imaging method, a CCD camera may, for example, be suitable as the examination instrument 7, the gates 6a, 6b being designed as light-tight doors. As soon as the living being 8 is present in the examination area 15, the examination is carried out for a predeterminable time period Δt. Such a time period may preferably be from 0.5 to 5 s, in particular about 1 s.

An advantageous use of the method, or of the device, serves to immobilize animals during optical in vivo domestic animal imaging. In this case, light in the infrared range is projected through a window 4 from a light source 1 and a light filter 2, onto the domestic animal which is inside the examination area 15 and has been provided with a contrast agent. The reflected light rays which, for example, denote fluorescent regions of the domestic animal 8, are detected by way of an optical CCD camera, which collects of the light rays through an image filter 9 and an image lens 10. The digital data of the CCD camera are sent to a corresponding image processing system 13. The lens 3 and the examination instrument 7 are fitted in a light-tight housing 14 by way of supports 11, and are accessible through a lid 12.

The domestic animal 8 is guided from a waiting area (for example a pen, not shown here) through the entry 5 into an introduction area 16a. As soon as a domestic animal 8 which has just been examined is inside the discharge area 16b, the second gate 6b is closed and the first gate 6a is opened. The domestic animal 8 which is inside the introduction area 16a can now enter the examination area 15. As soon as the domestic animal 8 is inside the examination area 15, the first gate 6a is closed and the examination is activated. While the animal in the opaque tunnel is passing through the measurement field of the optical instrument, that is to say is in the examination area 15, the analysis is carried out automatically. A high-sensitivity CCD camera allows exposure times of less than 1 s, so that corresponding movements of the domestic animal 8 interfere scarcely or not at all with the recording. Between the light-tight housing 14 and the examination area 15 there is, for example, a window 4 made of an NIR-transparent material.

As soon as the examination has been carried out, that is to say after the predeterminable time period Δt, the second gate 6b is opened. The domestic animal 8 then enters the discharge area 16b and can return via the exit 17 into the waiting area (not shown).

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. A method for examining a living being, the method comprising:
stabilizing the living being in an examination area, using at least two gates, for at least a predeterminable time period, wherein the examination area is operatively coupled to an examination instrument; and
automatically carrying out the examination of the living being, using the examination instrument, during the predeterminable time period.

2. The method as claimed in claim 1, wherein the examination area and the at least two gates are parts of an examination system, the examination system further including an entry, a discharge area and an exit.

3. The method as claimed in claim 1, further comprising the steps of:
closing a second of the at least two gates;
closing a first of the at least two gates as soon as the living being has entered the examination area; and
opening the second gate after having closed the first gate, and after the predeterminable time period has elapsed.

4. The method as claimed in claim 1, wherein the examination is started as soon as the living being is in the examination area.

5. A device for stabilizing and examining a living being, comprising:
an examination area; and
an examination instrument, operatively coupled to the examination instrument, wherein the living being is examined during a predeterminable time period and is enclosable in the examination area during at least the predeterminable time period, and wherein an examination of the living being is carried out automatically by the examination instrument during the predeterminable time period.

6. The device as claimed in claim 5, wherein at least two gates are adapted to enclose the living being in the examination area.

7. The device as claimed in claim 5, wherein an entry, the examination area and an exit form an elongate tunnel, including a diameter that substantially restricts the freedom of movement of the living being other than along the tunnel.

8. The device as claimed in claim 5, wherein the examination area and the at least two gates are opaque, and wherein the examination instrument is an optical examination instrument, operatively coupled to the examination area via a window.

9. The device as claimed in claim 6, wherein the examination instrument is a CCD camera, the predeterminable time period is from 0.5 to 5 seconds, and the at least two gates are light-tight doors.

10. The method as claimed in claim 1, wherein the method is for stabilizing animals during optical in vivo domestic animal imaging.

11. The device as claimed in one of the preceding claims, characterised in that the device is used for immobilizing animals during optical in vivo domestic animal imaging.

12. The method as claimed in claim 2, further comprising the steps of:
closing a second of the at least two gates, relatively closest to the exit;
closing a first of the at least two gates, relatively closest to the entry, as soon as the living being has entered the examination area; and
opening the second gate after having closed the first gate, and after the predeterminable time period has elapsed.

13. The device as claimed in claim 6, wherein an entry, the examination area and an exit form an elongate tunnel, including a diameter that substantially restricts the freedom of movement of the living being other than along the tunnel.

14. The device as claimed in claim 6, wherein the examination area and the at least two gates are opaque, and wherein the examination instrument is an optical examination instrument, operatively coupled to the examination area via a window.

15. The device as claimed in claim 7, wherein the examination area and the at least two gates are opaque, and wherein the examination instrument is an optical examination instrument, operatively coupled to the examination area via a window.

16. The device as claimed in claim 13, wherein the examination area and the at least two gates are opaque, and wherein the examination instrument is an optical examination instrument, operatively coupled to the examination area via a window.

17. The device as claimed in claim 7, wherein the examination instrument is a CCD camera, the predeterminable time period is from 0.5 to 5 seconds, and the at least two gates are light-tight doors.

18. The device as claimed in claim 8, wherein the examination instrument is a CCD camera, the predeterminable time period is from 0.5 to 5 seconds, and the at least two gates are light-tight doors.

19. The device as claimed in claim 13, wherein the examination instrument is a CCD camera, the predeterminable time period is from 0.5 to 5 seconds, and the at least two gates are light-tight doors.

20. The device as claimed in claim 14, wherein the examination instrument is a CCD camera, the predeterminable time period is from 0.5 to 5 seconds, and the at least two gates are light-tight doors.

21. The device as claimed in claim 15, wherein the examination instrument is a CCD camera, the predeterminable time period is from 0.5 to 5 seconds, and the at least two gates are light-tight doors.

22. The device as claimed in claim 16, wherein the examination instrument is a CCD camera, the predeterminable time period is from 0.5 to 5 seconds, and the at least two gates are light-tight doors.

23. The method of claim 1, wherein the predeterminable time period is from 0.5 to 5 seconds.

24. The method of claim 1, wherein the predeterminable time period is less than 1 second.

25. The device as claimed in claim 5, wherein the predeterminable time period is less than 1 second.

26. A system for examining a living being, comprising:
means for stabilizing the living being in an examination area for at least a predeterminable time period, wherein the examination area is operatively coupled to an examination instrument; and
means for automatically carrying out the examination of the living being during the predeterminable time period.

27. The system as claimed in claim 26, wherein the examination area and the means for stabilizing are parts of an examination system, the examination system further including an entry, a discharge area and an exit.

28. The system as claimed in claim 26, wherein the means for stabilizing includes at least two gates and wherein a second of the at least two gates is first closed, a first of the at least two gates is then closed as soon as the living being has entered the examination area, and the second gate is then opened after having closed the first gate, and after the predeterminable time period has elapsed.

29. The system of claim 26, wherein the predeterminable time period is from 0.5 to 5 seconds.

30. The system of claim 26, wherein the predeterminable time period is less than 1 second.
